(19) United States
(12) Patent Application Publication
(10) Pub. No.: US 2024/0382566 A1
(43) Pub. Date: Nov. 21, 2024

(54) A GLUCOSE-SENSITIVE INSULIN-LOADED MICROPARTICLE

(71) Applicants: FONDAZIONE ISTITUTO ITALIANO DI TECNOLOGIA, Genova (IT); THE BOARD OF TRUSTEES OF THE LELAND STANFORD JUNIOR UNIVERSITY, Stanford, CA (US); IRCCS "OSPEDALE POLICLINICO SAN MARTINO", Genova (IT)

(72) Inventors: Rosita PRIMAVERA, Genova (IT); Paolo DECUZZI, Bari (IT); Avnesh S. THAKOR, Palo Alto, CA (US); Angelo DE PASCALE, Genova (IT)

(21) Appl. No.: 18/578,830

(22) PCT Filed: Jul. 11, 2022
(86) PCT No.: PCT/EP2022/069278

§ 371 Date: Jan. 12, 2024

(30) Foreign Application Priority Data
Jul. 12, 2021 (IT) 102021000018221

(51) Int. Cl. A61K 38/28; A61K 9/50; A61P 3/10

(52) U.S. Cl. CPC A61K 38/28; A61K 9/5031; A61P 3/10

(57) ABSTRACT

A glucose-sensitive insulin-loaded microparticle which has a polymeric porous matrix made of a biocompatible and biodegradable polymer, insulin granules encapsulated within the polymeric porous matrix, and a blood glucose sensor attached to a surface of the glucose-sensitive insulin-loaded microparticle is provided. Use of the glucose-sensitive insulin-loaded microparticle for treatment of diabetes and for controlling glucose levels in the blood of a subject is also provided.

### FIELD OF THE INVENTION

This invention relates generally to a drug delivery system for the sustained and controlled release of insulin to a subject over a prolonged period of time. The invention also relates to the use of the insulin delivery system for the therapeutic treatment of diabetes and for controlling blood glucose levels in a subject in need thereof.

### BACKGROUND OF THE INVENTION

Insulin therapy is considered the primary approach for treating type-1 diabetes (T1D) and is also sometimes required in cases of advanced type-2 diabetes (T2D). Insulin is a dipeptide endocrine hormone secreted by pancreatic B-cells which are present in pancreatic islets. It consists of a total of 51 amino acids with a molecular weight of 5.8 kDa and is made of two chains which are inter-linked by disulphide bridges. [1] Insulin is a key molecule that regulates the intracellular transport of glucose into insulin-dependent tissues, such as adipose tissue, skeletal muscle and liver. It binds to specific insulin receptors located on the outer membrane of target cells, which then results in the activation of insulin signaling pathway resulting in the recruitment and translocation of the glucose transporter type 4 (GLUT4) to the cell membrane. [2]

Before 1980s, insulin was obtained from porcine or bovine pancreas for clinical applications. Nowadays, insulin can be synthesized, by recombinant DNA technology, in a form which is chemically identical to human insulin. Insulin preparations currently on the market include rapid-acting (insulin lispro, insulin aspart and insulin glulisine), intermediate-acting (neutral protamine Hagedorn (NPH) insulin and insulin Lente) and long-acting (Ultralente, insulin glargine, insulin detemir) formulations. [3, 4]

Insulin is conventionally administered via subcutaneous injections at various sites, i.e. upper limbs, tights, buttocks and abdomen, using syringes or pens or intravenous infusion for emergencies. Unfortunately, subcutaneous administration is associated with pain and poor patient's compliance due to various factors such as needle phobia, skin bulges, allergic reactions, common infections, stress generated from the difficult long-term regimen of insulin therapy. Furthermore, acute complications may occur from insulin overdose, where people can become hypoglycemic that, if severe, can result in coma, brain damage and death. Inadequate glucose control can also result in fluctuations in blood glucose levels (BGLs) that, if chronic, can increase the risk of developing cardiovascular disease, stroke, non-healing wounds, retinopathy (blindness), cancer, nephropathy, neuropathy, and many other co-morbidities.

In order to improve the quality of life of diabetic patients, different approaches have been explored with the aim to control and facilitate insulin delivery. Various formulations (i.e. oral or nasal spray or inhales insulin preparations) have been evaluated in preclinical and clinical trials. However, the poor permeability of insulin across the physiological barriers, the low bioavailability of insulin due to chemical and enzymatic degradation after interaction with the physiological environment (such as the harsh environment of the gastrointestinal tract), make it challenging to obtain an optimal formulation capable of matching the needs of diabetic patients.

Micro and nanoscale delivery vehicles are known to drastically improve the pharmacological properties (e.g. solubility, circulation half-life and toxicity) of encapsulated drugs, thereby leading to safer and more efficient treatments. The prior art describes encapsulating drugs into specific organic or inorganic macro- or nano-delivery platforms, in order to provide the following advantages [5, 6]:
(a) enhancing the therapeutic efficacy and minimize adverse effects of the drug;
(b) preventing premature drug degradation and denaturation; and
(c) enhancing the uptake of the drug through the physiological barriers (i.e. the skin and intestinal wall mucosae).

Additionally, various glucose-responsive insulin delivery systems, consisting of a blood glucose (BGL) sensor (i.e. glucose oxidase (GOx); boronic acid derivatives such as phenylboronic acid (PBA); and glucose-binding protein like ConcavalinA (ConA) connected to insulin pumps are disclosed in the prior art but their use is limited by the inaccurate BGL feedback, biofouling and low patient compliance [7-9].

In previous papers by the present inventors [10, 11], hierarchical polymeric microparticles (designated as “μPLs”) were manufactured using a top-down fabrication approach. This approach allows to control precisely and independently the geometrical, mechanical and biopharmacological properties of μPLs. μPLs have been made with PLGA and have a square base with an edge length of 20 μm and a height of 5 μm. In these papers, the authors have shown that:
μPLs can be used as a drug depot, combining sustained release profile with a precise control in geometrical and mechanical properties, fostering optimal tissue implantation;
μPLs with different mechanical stiffness and release profiles can be realized by modulating the amount of PLGA, without affecting the μPL geometry;
μPLs can be loaded with small molecules and nanoparticles, thus realizing a multiscale, hierarchical system;
the release profiles of drugs and nanoparticles could be further modulated by changing the polymer properties and including stimulo-responsive molecules.

The abstract [20] describes a delivery system for the controlled release of insulin and the regulation of glycaemia. The delivery system is made of nanoscale granules of insulin encapsulated within a porous matrix made of a biodegradable polymer, i.e. poly (lactic-co-glycolic acid) (PLGA). In particular, the insulin granules were obtained with an accurate crystallization process resulting in spherical nanoparticles with a diameter of 209.9±15.8 nm. μPLs were prepared by a top-down approach resulting in 20×5 μm square microparticles made of PLGA.

Although preliminary data collected on a mice model may suggest that the described delivery system could be effective in modulating glucose levels, there is still a need for an insulin delivery system that provides controlled, long-term release of insulin and allows accurate control of blood glucose levels (BGL) eliminating or substantially reducing BGL fluctuations.

### DESCRIPTION OF THE INVENTION

To meet these and other needs, the present invention provides a glucose-sensitive insulin-loaded microparticle comprising:
(a) a pH-sensitive polymeric porous matrix made of a biocompatible and biodegradable polymer,
(b) insulin granules encapsulated within the polymeric porous matrix, and
(c) a blood glucose sensor attached to a surface of the microparticle.

As used herein, “insulin-loaded microparticle” means that the microparticle of the invention contains insulin, as explained in more detail under item (b) above and in further detail in the following description.

In a preferred embodiment, the pH-sensitive polymeric porous matrix of the microparticle of the invention is made of a biocompatible and biodegradable polymer selected from the group consisting of poly(lactic-co-glycolic) acid (PLGA), polyethylene glycol (PEG), hyaluronic acid, chitosan, polymethyl methacrylate, polylactic acid, polyglycolic acid, polycaprolactone, and any combination thereof. The most preferred polymer is poly(lactic-co-glycolic) acid (PLGA).

In another preferred embodiment, the blood glucose sensor is glucose oxidase (GOx) or phenylboronic acid (PBA). Preferably, the blood glucose sensor is attached to an outer surface of the polymeric porous matrix of the microparticle.

GOx is the most commonly used glucose sensor because of its high affinity for glucose. GOx is a homodimer composed of two identical 80 kDa subunits, highly specific for glucose. GOx catalyzes the oxidation of glucose to gluconolactone, which is then hydrolyzed to gluconic acid to generate hydrogen peroxide in the presence of oxygen.

Due to the presence of GOx, the pH-sensitive polymeric matrix of the microparticle of the invention is degraded upon local acidification in response to elevated glucose levels, thus triggering the release of insulin. Based on this strategy, the degradation rate of the pH-sensitive polymeric matrix of the microparticle of the invention varies proportionally to blood glucose levels.

Alternatively, phenylboronic acid (PBA) may be used as the blood glucose sensor, due to its affinity for diol-containing molecules. Accordingly, the PBA-functionalized polymeric porous matrix of the microparticle of the invention can bind glucose reversibly and this binding drives the swelling of the matrix, thus improving insulin release. Based on this strategy, the microparticle of the invention can work as an artificial B-cell, fulfilling the insulin needs of a patient for several weeks.

The size, shape, surface properties and mechanical stiffness of the microparticle of the invention may be modulated by changing the amount of the biodegradable and biocompatible polymer making up the pH-sensitive polymeric porous matrix.

In a preferred embodiment, the microparticle of the invention is a microplate having a polygonal or substantially round base. Even more preferably, the microplate base is substantially square or rectangular in shape.

In general, the shape of a microparticle of the invention can be modulated and precisely defined during the fabrication process [10, 11].

In the case of a microplate having a polygonal base, the edge length preferably varies from 1 to 100 μm and the height preferably varies from 1 to 100 μm. Even more preferably, the edge length varies from 5 to 50 μm and the height varies from 5 to 20 μm.

Preferred dimensions for a microplate having a square base are an edge length of 20 μm and a height of 5 to 10 μm, more preferably a height of 10 μm.

The insulin granules which are uniformly dispersed in the polymeric porous matrix of the microparticle of the invention have a diameter preferably comprised between 50 nm to 500 nm, more preferably between 100 and 200 nm. A particularly preferred diameter is of 200 nm.

The modular engineering of the microparticle's size and shape and insulin granule size allows the design of patient-specific, minimally invasive insulin depots. Specifically, the micrometric size of the microparticle of the invention allows for a sustained release of insulin over weeks, while the precise control in geometrical and mechanical properties foster optimal implantation. Indeed, smaller insulin-nanoparticles [18] can only release the active principle for a short period of time (hours), thus requiring multiple weekly injections to control BGL. On the other hand, macroscopic drug depots (e.g. insulin-pumps of the prior art) are invasive for the patient.

The insulin granules which are dispersed in the polymeric porous matrix provide a method of delaying the action of insulin, which is very similar to the method used by the pancreas for the storage and/or release of insulin. Insulin in fact is an anabolic hormone which is stored in pancreatic β-cells as granules consisting of insoluble crystalline hexameric insulin. [12] The secretion of insulin from β-cells is stimulated by elevated exogenous glucose levels, such as those occurring after a meal [13, 14]. After secretion, the natural granules of insulin dissolve rapidly in the bloodstream and, after docking to its receptor on muscle and/or adipose tissue, insulin enables the insulin-dependent uptake of glucose into these tissues and reduces BGLs by removing the exogenous glucose from the bloodstream. Likewise, the glucose-sensitive insulin-loaded microparticle of the invention dissolves easily when in contact with a physiological solution such as PBS at neutral pH, favoring a rapid release of insulin.

Additionally, the glucose-sensitive insulin-loaded microparticle of the invention exhibits a good stability profile (30 days), without any alteration in insulin function, as demonstrated by quantifying the amount of AKT phosphorylation caused by the activation of the insulin receptor. It is worth to note that insulin stability is one of the major causes which limit the encapsulation of insulin in biodegradable polymeric microparticles obtained using conventional bottom-up fabrication process (i.e. emulsion/solvent removal technique) [15-17].

In summary, the microparticles of the invention have the following advantages:
They are minimally invasive insulin-depot, as compared to macroscopic pumps;
They allow for a long term (weeks) sustained release of insulin, as compared to faster release from insulin nanoparticles;
They comprise a blood glucose sensor directly integrated in the polymeric matrix of the depot;
They comprise a hydrophobic polymeric network that limits water access and preserves INS-granules functionality over weeks;
They are manufactured by a top-down fabrication approach, which avoids the use of extra organic solvents and elevated temperatures and allows to maintain the bioactivity of insulin;
They exhibit a good stability profile (30 days) without any alteration in insulin function.

In light of the above-illustrated properties and advantages, microparticles according to the invention are particularly suitable for the preparation of a sustained drug delivery system capable of releasing insulin in the blood of a subject in need thereof over a prolonged period of time. Such a drug delivery system is suitable for use in the therapeutic treatment of diabetes (i.e. either type-1 diabetes (T1D) or type-2 diabetes (T2D)) and/or for controlling glucose levels in the blood of a subject in need thereof.

The sustained drug delivery system of the invention may be administered by any administration route, but oral administration is preferred.

Microparticles according to the present invention were synthesized and pre-clinically validated for the sustained and controlled release of insulin, as disclosed in the following examples.

The examples refer to the appended drawings, in which:

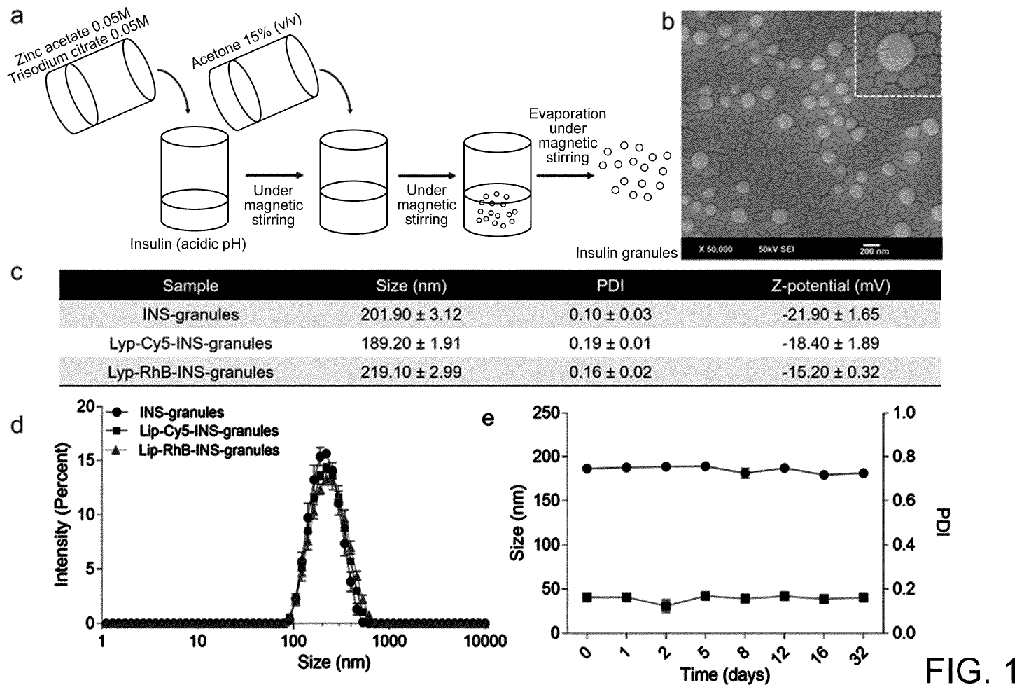

FIG. 1 shows the synthesis, physico-chemical characterization and stability of INS-granules. a. Schematic of the INS-granules crystallization process. b. HR-SEM image of INS-granules. c-d. Size distribution, polydispersity index (PDI) and surface charge of INS-granules and fluorescent INS-granules (Lip-Cy5-loaded INS-granules and Lip-RhB-loaded INS-granules). e. INS-granules stability in water at 25° C. for 31 days.

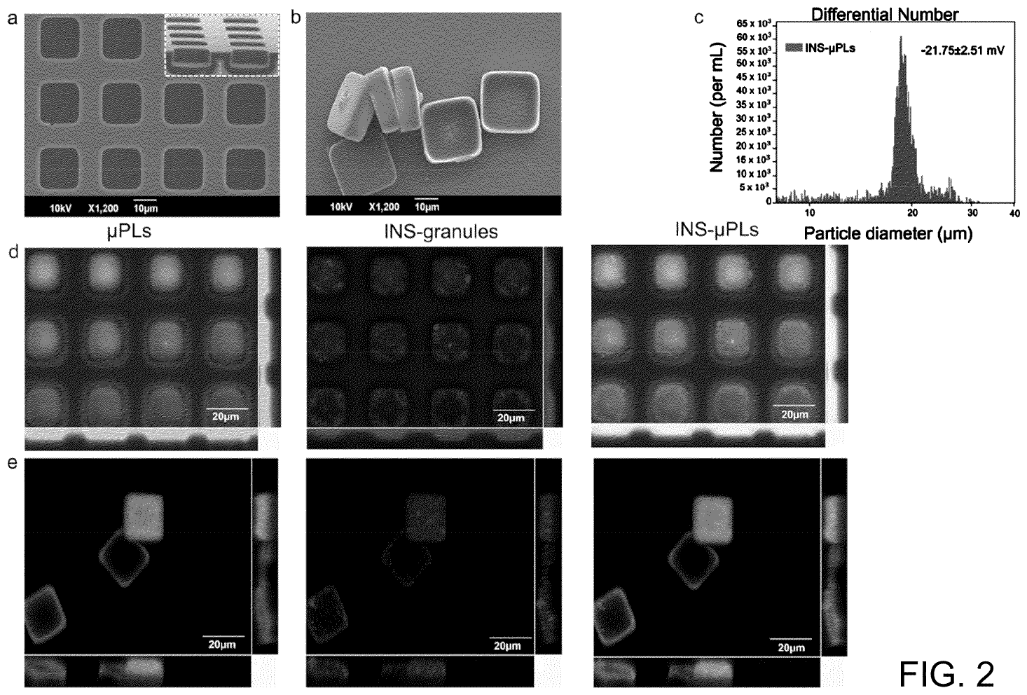

FIG. 2 shows the physico-chemical characterization of insulin-loaded microparticles (INS-μPLs). a,b. SEM images of PVA template (insert indicates the cross-section of PVA template) and INS-μPLs showing the characteristic 20×20×10 μm square shape. c. Size distribution and zeta-potential of INS-μPLs via Multisizer and dynamic light scattering analysis, respectively. d. Confocal images of RhB-stained PVA templates (blue) loaded with the polymeric mixture containing Lip-Cy5-INS-granules (red) and curcumin-stained PLGA (green). e. Confocal images of INS-μPLs obtained using curcumin-stained PLGA matrix (green) and loaded with Lip-Cy5-INS-granules (red).

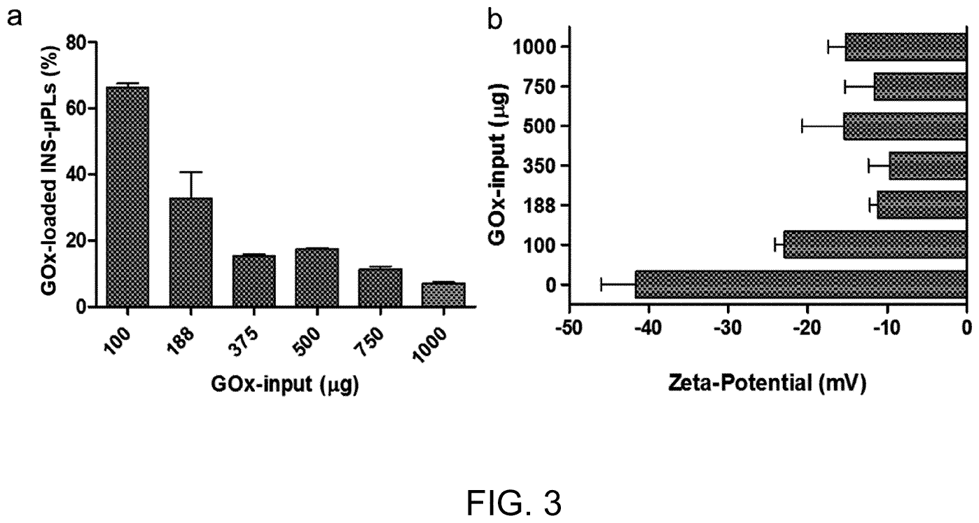

FIG. 3 shows the pharmaceutical characterization of glucose-sensitive INS-μPLs. a. Glucose oxidase loading efficiency of μPLs considering different initial input; b. Surface charge of GOX-loaded μPLs for different GOx initial input (100-1000 μg). Data points represent mean±SD (n=3).

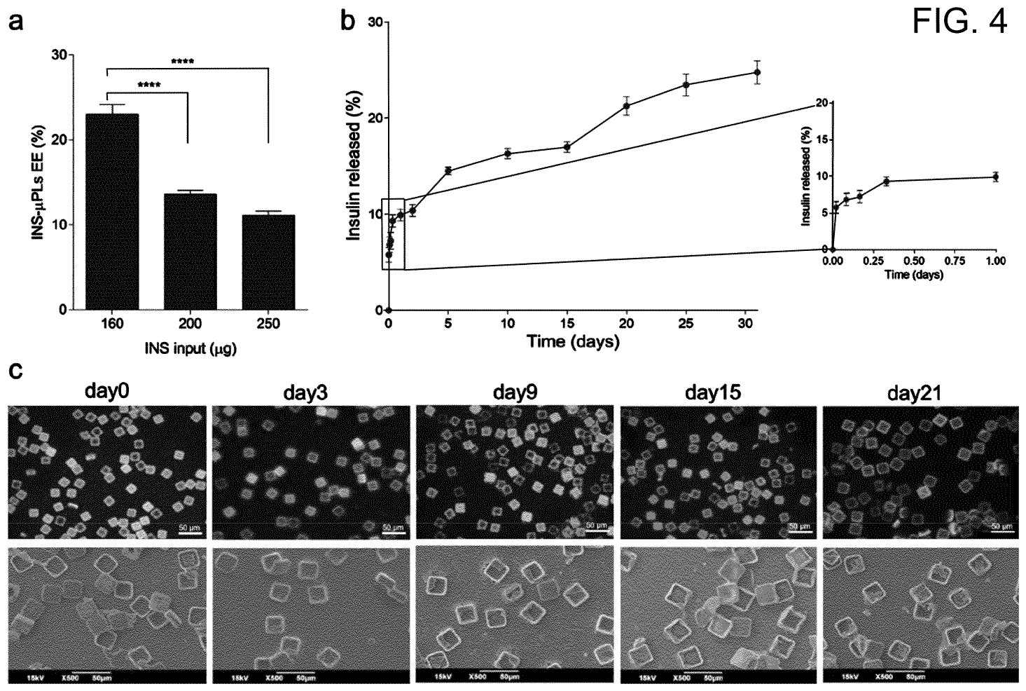

FIG. 4 shows the loading, release profile and stability of INS-μPLs. a. Encapsulation efficiency percentage (EE, %) of INS-μPLs using different INS-granules initial input (160, 200 and 250 μg) (n=5). b. In vitro insulin release profile of INS-μPLs in physiological condition (PBS, pH=7.4 at 37° C.) (n=3). c. Fluorescence microscopy and SEM images of μPLs in physiological condition (PBS, (pH=7.4) at 37° C.) overtime (n=3), which prove the stability of particles over 21 days of incubation. Results are expressed as average±SEM. Statistical significance was determined by One-way ANOVA post-hoc Tuckey Test. a) **** represents p<0.0001 for 160 μg vs. 200 μg and 160 μg vs. 250 μg.

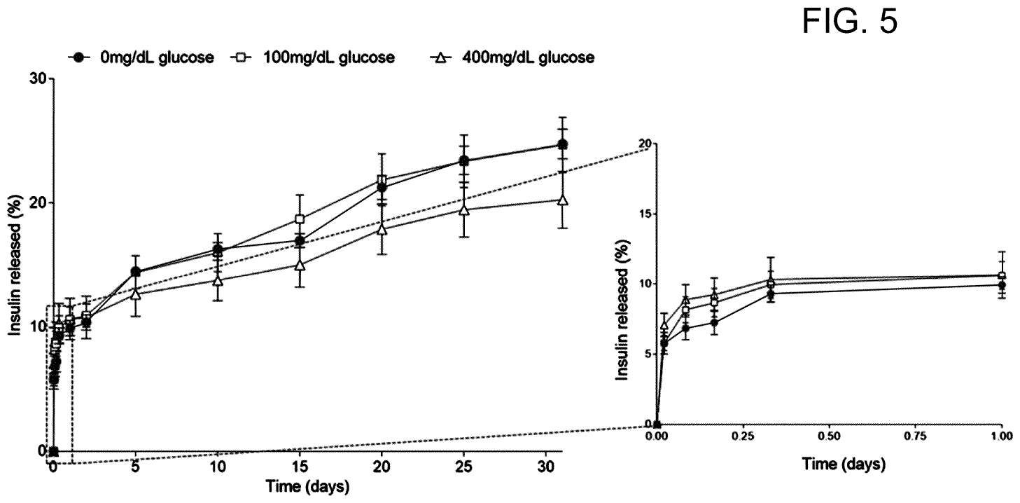

FIG. 5 shows the in vitro insulin release profile of the glucose-sensitive INS-μPLs in physiological condition (PBS, pH=7.4 at 37° C.) at different glucose concentrations: 0 mg/dL, 100 mg/dL (normoglycemia condition) and 400 mg/dL of glucose (hyperglycemia condition).

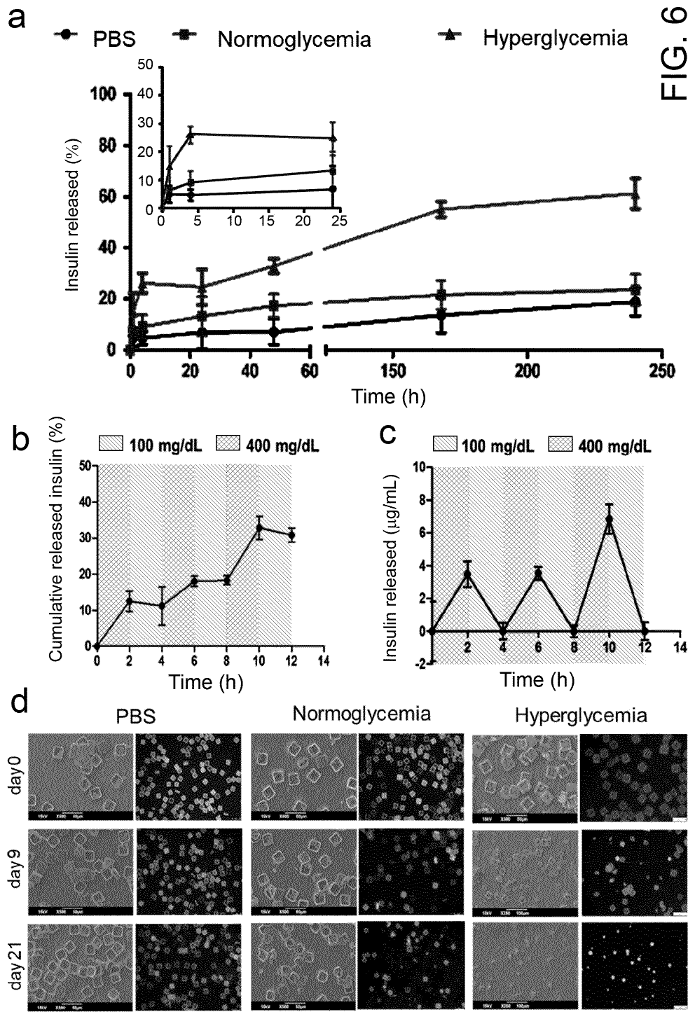

FIG. 6 shows insulin release profiles and glucose-responsive degradation of glucose sensitive INS-μPLs. a. In vitro insulin release of particles in different glucose concentrations in PBS: 0, 100 (normoglycemia), and 400 mg/dL (hyperglycemia) at 37° C.; b. Self-regulated profile of particles present the insulin released percentage as a function of glucose concentrations; c. Pulsatile release profile of particles present the rate of insulin release as a function of different glucose concentrations; d. Pictures of particles incubated in different glucose concentrations in PBS at 37° C. over time. Data points represent mean±SD (n=3).

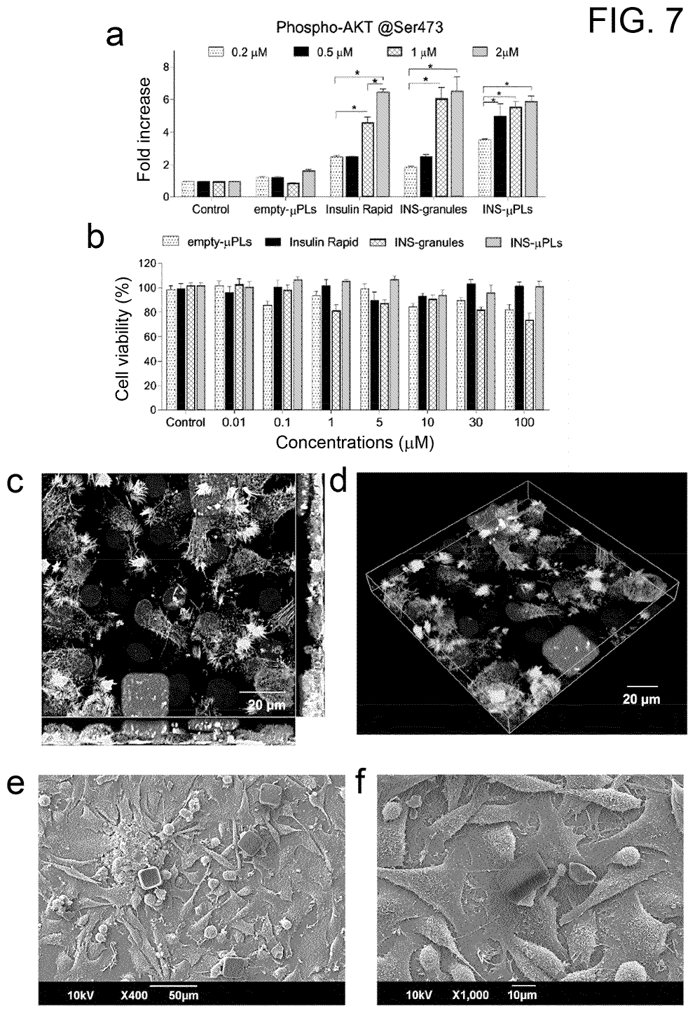

FIG. 7 shows the cytotoxicity and biological activity of INS-μPLs. a. Cytotoxic effect of INS-μPLs at different concentrations (0.01-100 μM) assessed on L6 cells. b. Biological activity of INS-μPLs on L6 cells, which prove the activation of insulin receptor through the phosphorylation of AKT at Ser473. c-d. 2D and 3D confocal microscopy images of L6 cells (blu=DAPI, green=phalloidin) after 24 h incubation with Lip-RhB-INS-granule-loaded μPLs, which shows that the safety of INSμPLs. e-f. SEM images of INS-μPLs after 24 of incubation with L6 cells. Results are expressed as the average±SEM (n=5). Statistical significance was determined by One-way ANOVA post-hoc Tuckey Test.

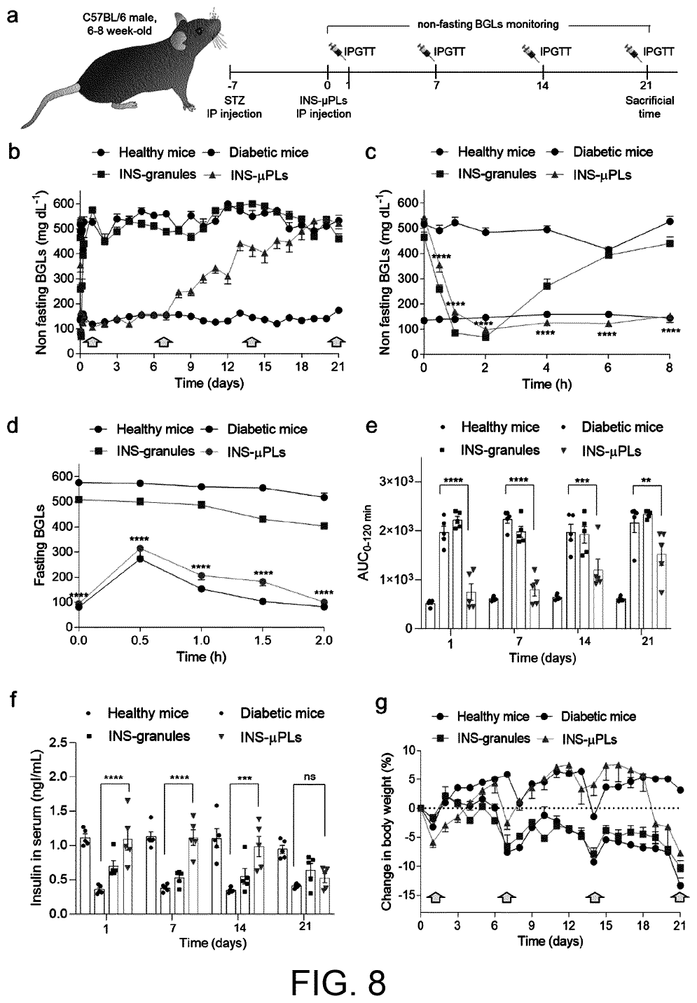

FIG. 8 shows the in vivo evaluation of INS-μPLs. a. Experimental setup for in vivo experiments in C57BL/6 STZ-induced diabetic mice (yellow arrows indicate the IPGTT). b. Non-fasting blood glucose measurements over 21 days after intraperitoneal (IP) injection of INS-μPLs. c. Non-fasting blood glucose measurements over the first 8 h after intraperitoneal (IP) injection of INS-μPLs. d. Fasting Intraperitoneal Glucose Tolerance Test (IPGTT) at day 7 post-injection of INS-μPLs. e. Responsiveness in diabetic mice was calculated based on the area under the curve (AUC_{0-120 min}) from 0-120 min at different days (1, 7, 14, 21 days after ip injection of INS-μPLs). f. Change in body weight (%). g. Serum insulin levels at different days (1, 7, 14, 21 days after intraperitoneal injection of INS-μPLs). Results are expressed as the average±SEM (n=5). Statistical significance was determined by Two-way ANOVA post-hoc Tuckey Test. c, d) **** represents p<0.001 for INS-μPLs vs. diabetic mice.

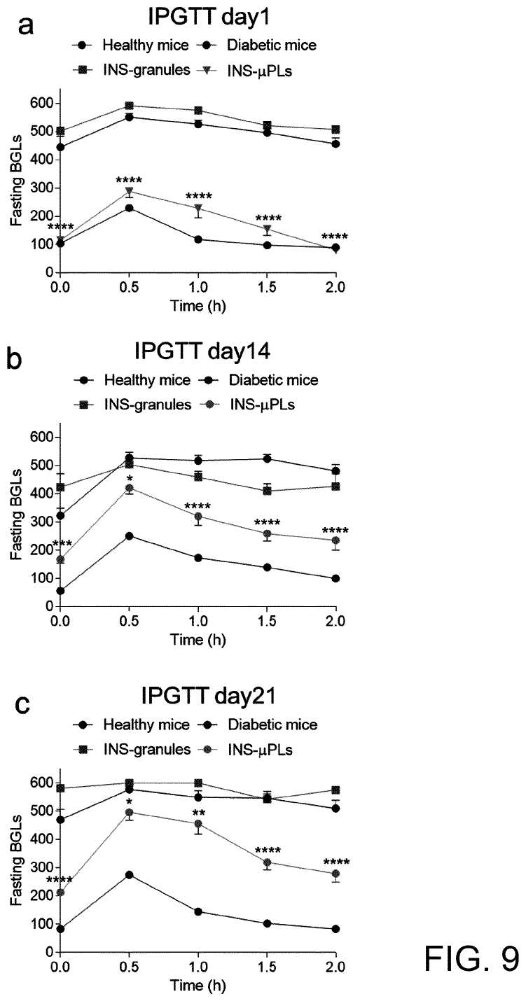

FIG. 9 shows the Intraperitoneal Glucose Tolerance Test (IPGTT) after INS-μPLs injection overtime: a. day 1, b. day 14, c. day 21. Results are expressed as the average±SEM (n=5). Statistical significance was determined by Two-way ANOVA post-hoc Tuckey Test. b) * represents p<0.05, ** represents p<0.01, *** represents p<0.001 and **** represents p<0.0001 of INS-μPLs vs. diabetic mice.

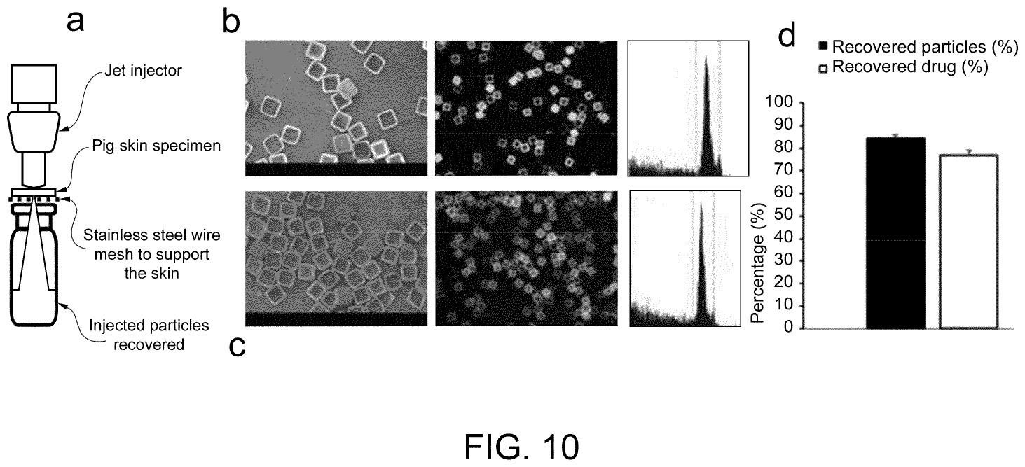

FIG. 10 shows the INS-μPLs delivery through the skin using a needle-free jet injector a. Schematic representation of the experimental setup, b-c. CURC-loaded INS-μPLs before (b) and after (c) the passage through pig skin specimen (the average size, shape and number of particles were determined using scanning electron microscopy and fluorescence microscopy), d. Recovered drug and particles after the passage through the skin, expressed as % of an un-injected control. The amount of curcumin and the number of particles were determined by HPLC and multi-sizer particle counter, respectively.

The examples are provided for illustration purposes only and are not intended to limit the scope of the invention as defined by the appended claims.

### EXAMPLES

### Example 1: Synthesis of Glucose-Sensitive Insulin-Loaded Microparticles (Glucose-Sensitive INS-μPLs)

Insulin granules (INS-granules) were synthetized and encapsulated within the porous matrix of the microparticles (INS-μPLs). Then, INS-μPLs were integrated with glucose oxidase for a controlled release of insulin and the regulation of glycaemia.

### 1.1 Synthesis of Insulin Granules (INS-Granules)

INS-granules were prepared using a crystallization process as reported in the literature with some modifications. [19]

Briefly, insulin (5 mg mL⁻¹) was dissolved in acidified water (HCl 10 mM, pH=2.5), whereupon zinc acetate (0.05 M), trisodium citrate (0.05 M) and acetone 15% were added at room temperature under magnetic stirring (250 rpm) for 90 min. After the evaporation of the organic solvent, particles were collected by centrifugation 18,000 g for 30 min, washed in water and stored at 4° C. Fluorescent INS-granules were obtained using two different fluorescent probes: Lip-Cy5 and Lip-RhB to obtain Lip-Cy5-loaded INS-granules (Lip-Cy5-INS-granules) and Lip-RhB-loaded INS-granules (Lip-RhB-INS-granules), respectively.

1.2 Synthesis of Microparticles Loaded with Insulin Granules (INS-μPLs)

The synthesis of the polymeric microparticles follows a multistep process based on a template strategy, partially described in previous papers by the present inventors. [10, 11] In the first step, a silicon master template with a specific geometrical feature was fabricated using direct laser writing. The silicon master template had squared wells with an edge length of 20 μm and a depth of 10 μm, separated by a 10 μm gap. Then, the original master template was replicated into a polydimethylsiloxane (PDMS) template by covering it with a mixture of PDMS and a silicone elastomer curing agent (10:1, v/v). The silicon template with PDMS solution was left under vacuum to remove bubbles formed during the mixing process and was polymerized at 60° C. for 4 h. PDMS template was peeled off the silicon substrate and the resulting replica showed opposite geometrical pattern (pillars rather than wells) compared to silica template. PDMS template was used to obtain a poly(vinyl alcohol) (PVA) template by putting a PVA solution [10 w/v % in deionized (DI) water] on its patterned surface. After drying (60° C.), the PVA reproduces the original geometrical pattern of the master template (wells). In the last step, PVA template was loaded with the polymeric paste (usually, poly (lactic-co-glycolic) acid or other relevant polymers (i.e. polyethylene glycol, PEG] or their combination) and insulin granules (200 μg) were dissolved in acetonitrile (ACN), to generate INS-μPLs. After solvent evaporation, the loaded PVA template was dissolved in DI water at room temperature in an ultrasonic bath. PVA solution was removed by using polycarbonate membrane filters (50 μm pore size) and INS-μPLs are recovered through sequential centrifugation steps (5,000 rpm for 5 min).

### 1.3 Synthesis of Glucose-Sensitive INS-μPLs

### 1.3.1 GOx-INS-μPLs

The glucose sensor (i.e. glucose oxidase, GOX) was cross-linked on the surface of INS-μPLs.

GOx-INS-μPLs were prepared by a coupling reaction between the carboxyl groups on the surface of PLGA-μPLs and the amino groups of GOX in the presence of EDC (ethylene dichloride) and NHS (N-Hydroxysuccinimide). Briefly, INS-μPLs were dispersed in distilled water and EDC/NHS were added in a molar ratio of 1:3 (PLGA: EDC/NHS) for 3 h under rotation (1000 riv/min) at room temperature. After 3 hours of activation, the activators were removed through sequential centrifugation steps (5,000 rpm for 10 min). Then, GOx (2.5 mg/mL) was slowly added. The reaction mixture was maintained at 4 C° for 2 h. The activators and the excess of GOx were removed through sequential centrifugation steps (5,000 rpm for 10 min). Reaction time, GOx concentration, PLGA: EDC/NHS ratio of the coupling reaction between the carboxyl groups on the surface of PLGA-μPLs and the amino groups of GOX in the presence of EDC and NHS were carefully optimized. For example, a longer reaction regime affected negatively the INS load.

### 1.3.2 APBA-INS-μPLs

The PBA-surface-modified-μPLs was prepared by a coupling reaction between the carboxyl groups on the outer surface of PLGA-μPLs and amino group of APBA in the presence of EDC and NHS. Briefly, PLGA μPLs (0.2 g, about 0.01 mmol PLGA), EDC (0.03 mmol) and NHS (0.03 mmol) were dispersed in distilled water (30 mL). After four hours of activation, 3-aminophenylboronic acid (APBA, 0.02 mmol) was slowly added. The reaction mixture was incubated at 4° C. for 8 h in an ice bath. The PBA-μPLs were collected by centrifugation, repeated wash by distilled water under sonication conditions.

### Example 2: Characterization

### 2.1 Insulin-Granules

Insulin granules (INS-granules) were prepared using the crystallization process shown in FIG. 1a. INS-granules consist in a monodisperse population (polydispersity index (PI)=0.10±0.03) of spherical particles with an average size of 201.9±3.12 nm. Their spherical shape and the average size were confirmed by SEM analysis (FIG. 1b,c,d). INS-granules showed a net negative surface charge (−21.90±1.65 mV), thus demonstrating that INS-granules were stable for at least 30 days at room temperature in water (FIG. 1c,e). When a fluorescent probe such as Lip-Cy5 and Lip-RhB were added, INS-granules showed no significant difference in the average size (189.20±1.91 and 199.10±2.99 nm, respectively) and surface charge (−18.40±1.89 and −15.20±5.32 mV, respectively) (FIG. 1c,d).

### 2.2 INS-μPLs

After preparation, INS-granules were encapsulated within the poly (lactic-co-glycolic) acid porous matrix of biodegradable μPLs to provide μPLs-loaded with INS granules (i.e. INS-μPLs). INS-μPLs were obtained via replica molding multi-step, top-down fabrication process [11]. Briefly, a silicon master template was fabricated via Direct Laser Writing and replicated into a PDMS template, whose pattern was then transferred into a sacrificial PVA template. This PVA template was loaded with the polymeric paste (PLGA) and INS-granules constituting the final INS-μPLs. INS-μPLs were released and collected upon dissolution in water of the sacrificial PVA template. INS-μPLs were square in shape with an edge length of 20 μm and a height of 10 μm, precisely replicating the size and shape of the wells of their original silicon template configuration, as confirmed by SEM images of the cross-sectional and frontal view of the μPLs PVA template and particles (FIG. 2a,b). A multisizer analysis showed a single peak between 15 μm and 20 μm (FIG. 2c) in fact, given the non-spherical shape of the μPLs, the instrument returns an average characteristic size rather than the actual edge length of the particles [11, 20]. The μPL electrostatic surface charge was-21.75±2.51 mV, which is due to the carboxylic termination of the PLGA chains. The confocal analysis of the Lyp-Cy5-INS-granules (red) dispersed within the polymeric paste of μPLs (green) and μPLs' PVA template (blue) confirmed the actual shape and size of INS-μPLs and also showed INS-granules (red) quite homogenously dispersed inside the PLGA polymeric matrix (green) of the μPLs (FIG. 2d,e). Lip-Cy5-INS-granules encapsulated in green μPLs were obtained by loading Lip-Cy5-INS-granules in a polymeric paste containing PLGA and CURC within the sacrificial PVA template stained with RhB.

### 2.3 Glucose-Sensitive INS-μPLs

For evaluating the GOx binding on the u PLs surface after exposure at different GOx amounts (100-1000 μg), GOx quantification was performed via BCA quantification (protein quantification assay) and the GOx binding was confirmed through the evaluation of INS-μPLs surface charge (FIG. 3).

### Example 3: Degradation and Release of INS-μPLs

To assess insulin entrapment efficiency (EE %), INS-μPLs were synthetized using different INS-granules initial inputs (160, 200 and 250 μg) per PVA template. As reported in FIG. 3a, the EE % is higher using 160 μg (22.82±2.51%) of insulin compared to 200 and 250 μg (13.60±1.08 and 11.12±1.10%, respectively). Furthermore, insulin release profile was evaluated up to 1 month under physiologically relevant conditions in 0.5 mL of PBS (pH=7.4) at 37° C., mimicking the volume associated with an intra-tissue deposition of μPLs. Data showed a characteristic initial fast release of insulin during the first 24 h (9.93±1.05%), followed by a slower release profile over the remaining 30 days when only 24.78±2.07% of insulin were released (FIG. 4). Notice that, this phenomenon is due to the fact that in a small release volume (i.e. 0.5 mL), the drug concentration gradient between the μPLs and the surrounding solution rapidly decreases thus reducing the release rate. The release kinetics were then studied in response to elevated (400 mg dL⁻¹) or physiological (100 mg dL⁻¹) glucose concentrations, showing no significant variations in INS-μPLs release profile (FIG. 5). Moreover, to verify the matrix biodegradation, INS-μPLs stained with CURC (green) were synthetized and characterized over time (1 month) for possible morphological changes. SEM images taken at different time points upon INS-μPLs exposure to physiological conditions (0.5 mL of PBS, pH=7.4 at 37° C.) demonstrated the typical square shape of the μPLs for longer time points, proving that drug release profile is not affected from particles biodegradation but from the diffusion of the drug molecules from the matrix core to the surrounding aqueous environment (FIG. 4c).

### Example 4: Degradation and Release of Glucose-Sensitive INS-μPLs

To study the progressive release of insulin granules from the polymeric matrix of INS-μPLS, various solutions (0, 100 or 400 mg/dL glucose in PBS) were added to glucose-sensitive INS-μPLs and incubated at 37° C. in an orbital shaker to evaluate the release of insulin. At each time point, the solution was centrifuged at 3250 ref for 5 minutes. The supernatant was then removed and replaced with fresh PBS. The insulin content was analyzed by UPLC-MS and the concentration was interpolated from an insulin standard curve. (FIG. 6a). Furthermore, glucose-sensitive INS-μPLs were immersed into PBS buffer containing different concentrations of glucose each 2 h for assessing the dynamic response of glucose-sensitive INS-μPLs to glucose pool variation (FIG. 6b,c). For confirming the effect of glucose sensor, INS-μPLs were monitored and checked at SEM and fluorescent microscope after exposure at various glucose concentrations for at least 20 days (FIG. 6 d).

### Example 5: In Vitro Activity and Biocompatibility of INS-μPLs

To show if insulin remains bioactive after encapsulation in μPLs and following release, a cell-based assay was performed to quantify the amount of phosphorylated-AKT caused by the activation of the insulin receptor (FIG. 7a). Both INS-μPLs and INS-granules increased the phosphorylation of AKT and the effect resulted dose-dependent (p<0.05). INS-μPLs resulted more efficacious at lower concentration (0.5 μM) compared to a commercial form of insulin (Insulin Rapid) (5.00±1.3 vs. 2.52±0.1-fold increase, respectively). While, as expected any effect was showed using empty-μPLs. Furthermore, INS-μPLs showed no significant cytotoxic effect across the tested spectrum of concentrations (0.01-100 μM), and confocal and SEM images confirmed no effect on cell morphology for up to 24 h (FIG. 7b,c,d,e,f).

### Example 6: In Vivo Activity of INS-μPLs

To evaluate the ability of the INS-μPLs to provide glycemic control in vivo, we employed streptozotocin (STZ)-induced type 1 diabetic C57BL/6 mouse model. Diabetic mice were randomly grouped and intraperitoneally injected with either INS-granules and INS-μPLs at insulin dosage 0.05 IU g⁻¹ body weight and 0.5 IU g⁻¹ body weight, respectively. During the entire treatment (21 days), the BGLs of treated mice were monitored carefully by taking blood from the tail vein of the mice (FIG. 8a). As expected, the BGLs in mice treated with INS-u PLs and INS-granules were reduced at virtually the same BGLs as those in the healthy group at 1 h post-implantation (167.4±49.0 and 86.0±12.4 vs. 140.0±9.2 mg dL⁻¹. respectively), indicating a rapid onset of insulin release in elevated BGLs. However, the normoglycemic state could not be maintained in the groups treated with INS-granules, and the glucose levels returned to a hyperglycemic state after 6 h (393.4±71.6 mg dL⁻¹). In contrast, only one dose of INS-μPLs was required to provide glycemic control in vivo for almost two weeks, returning to hyperglycemic state (>350 mg dL⁻¹) 13 days post-implantation (441.6±66.8 mg dL⁻¹) (FIG. 8b,c). An intraperitoneal glucose tolerance test (IPGTT) was performed administering glucose at dosage 2 g kg⁻¹ in fasted mice at 1, 7, 14, 21 days post-administration of INS-μPLs to assess blood glucose regulation capacity. At day 1 and 7, diabetic mice treated with INS-μPLs successfully regained glycemic control after an initial spike in BGLs and maintained BGLs in the normoglycemic range (70-200 mg dL⁻¹), whereas mice were not able to regulate BGLs at 13 days post-injection as demonstrating by IPGTT at day 14 and 21 (FIG. 8d,e; FIG. 9). Likewise, diabetic mice treated with INS-μPLs are not showing significant difference in AUC_{0-120 min} compared to healthy mice at day 1 (749.2±167.4 vs. 522.6±27.6, respectively) and 7 (799.9±134.9 vs. 609.8±18.6, respectively), whereas a significant increase of AUC_{0-120 min} has been demonstrated at day 14 (1203.3±217.8 vs. 641.9±26.2, respectively; p<0.05) and 21 (1518.3±225.9 vs. 606.7±22.3, respectively; p<0.0001). Consistently with registered euglycemic levels, serum insulin measurement by an enzyme-linked immunosorbent assay (ELISA) exhibited a release of insulin in mice treated with INS-μPLs at day 1, 7 and 14 significantly higher compared to diabetic mice (1.10±0.16 vs. 0.35±0.03 ng mL⁻¹; 1.11±0.11 vs. 0.38±0.02 ng mL⁻¹; 0.98±0.15 vs. 0.34±0.02 ng mL⁻¹ at day 1, 7 and 14, respectively; p<0.0001), followed by a reduction in serum insulin at day 21 (0.52±0.07 vs. 0.41±0.14 ng mL⁻¹) (FIG. 8f). Furthermore, mice treated with INS-μPLs have been demonstrated no significant changes in body weight compared to healthy mice for at least 18 days of treatment. In contrast, diabetic mice and mice treated with INS-granules have been registered a significant weight loss starting from day 5 (p<0.05) (FIG. 8g).

### Example 7: INS-μPLs Delivery Through the Skin Using a Needle-Free Jet Injector

The design of INS-μPLs were also tested for the combined used with a commercial needle-free jet injector with the objective of realizing a sub-cutaneous drug depot of insulin with high patients' compliance. To this end, CURC-loaded INS-μPLs were injected through a porcine skin specimen (FIG. 10a). The shape, size, surface properties, mechanical stiffness and biodegradability of the developed particles make them ideal for tissue implantation and prolonged release of active molecules. The particle number, shape and integrity were assessed before and after the injection by multisizer particle counter, fluorescent microscopy and scanning electron microscopy. Furthermore, the amount of the recovered drug and particles after the passage through the skin, were expressed as % of an un-injected control. The amount of curcumin and the number of particles recovered were determined by HPLC and multisizer particle counter, respectively. Results showed no detrimental effect on particles morphology upon high velocity impact with the skin. (FIG. 10b, c, d).

### REFERENCES

[1] R. Primavera, B. D. Kevadiya, G. Swaminathan, R. J. Wilson, A. De Pascale, P. Decuzzi, A. S. Thakor, Emerging Nano- and Micro-Technologies Used in the Treatment of Type-1 Diabetes, Nanomaterials 10 (4) (2020) 789.
[2] I. B. Hirsch, Insulin analogues, New England Journal of Medicine 352 (2) (2005) 174-183.
[3] C. Homko, A. Deluzio, C. Jimenez, J. W. Kolaczynski, G. Boden, Comparison of insulin aspart and lispro: pharmacokinetic and metabolic effects, Diabetes care 26 (7) (2003) 2027-2031.
[4] M. Lepore, S. Pampanelli, C. Fanelli, F. Porcellati, L. Bartocci, A. Di Vincenzo, C. Cordoni, E. Costa, P. Brunetti, G. B. Bolli, Pharmacokinetics and pharmacodynamics of subcutaneous injection of long-acting human insulin analog glargine, NPH insulin, and ultralente human insulin and continuous subcutaneous infusion of insulin lispro, Diabetes 49 (12) (2000) 2142-2148.
[5] J. Shi, A. R. Votruba, O. C. Farokhzad, R. Langer, Nanotechnology in drug delivery and tissue engineering: from discovery to applications, Nano letters 10 (9) (2010) 3223-3230.
[6] D. Bobo, K. J. Robinson, J. Islam, K. J. Thurecht, S. R. Corrie, Nanoparticle-based medicines: a review of FDA-approved materials and clinical trials to date, Pharmaceutical research 33 (10) (2016) 2373-2387.
[7] N. A. Bakh, A. B. Cortinas, M. A. Weiss, R. S. Langer, D. G. Anderson, Z. Gu, S. Dutta, M. S. Strano, Glucose-responsive insulin by molecular and physical design, Nature chemistry 9 (10) (2017) 937-943.
[8] G. Chen, J. Yu, Z. Gu, Glucose-responsive microneedle patches for diabetes treatment, Journal of diabetes science technology 13 (1) (2019) 41-48.
[9] R. Mo, T. Jiang, J. Di, W. Tai, Z. Gu, Emerging micro- and nanotechnology based synthetic approaches for insulin delivery, Chemical society reviews 43 (10) (2014) 3595-3629.
[10] M. Di Francesco, R. Primavera, D. Romanelli, R. Palomba, R. C. Pereira, T. Catelani, C. Celia, L. Di Marzio, M. Fresta, D. Di Mascolo, D. Paolo, Hierarchical microplates as drug depots with controlled geometry, rigidity, and therapeutic efficacy, ACS applied materials interfaces 10 (11) (2018) 9280-9289.
[11] M. Di Francesco, R. Primavera, M. Summa, M. Pannuzzo, V. Di Francesco, D. Di Mascolo, R. Bertorelli, P. Decuzzi, Engineering shape-defined PLGA microPlates for the sustained release of anti-inflammatory molecules, Journal of Controlled Release 319 (2020) 201-212.
[12] Y. V. J. E. Li, Zinc and insulin in pancreatic beta-cells, 45 (2) (2014) 178-189.
[13] P. V. Röder, B. Wu, Y. Liu, W. J. E. Han, m. medicine, Pancreatic regulation of glucose homeostasis, 48 (3) (2016) e219-e219.
[14] M. Komatsu, M. Takei, H. Ishii, Y. J. J. o. d. i. Sato, Glucose-stimulated insulin secretion: A newer perspective, 4 (6) (2013) 511-516.
[15] H. J. P. j. o. p. s. Sah, technology, Protein instability toward organic solvent/water emulsification: implications for protein microencapsulation into microspheres, 53 (1) (1999) 3-10.
[16] W. Lu, T. G. J. P. J. o. P. S. Park, Technology, Protein release from poly (lactic-co-glycolic acid) microspheres: protein stability problems, 49 (1) (1995) 13-19.
[17] Y. Yeo, K. J. A. o. p. r. Park, Control of encapsulation efficiency and initial burst in polymeric microparticle systems, 27 (1) (2004) 1.
[18] L. R. Volpatti, M. A. Matranga, A. B. Cortinas, D. Delcassian, K. B. Daniel, R. Langer, D. G. J. A. n. Anderson, Glucose-responsive nanoparticles for rapid and extended self-regulated insulin delivery, 14 (1) (2019) 488-497.
[19] J. J. A. c. s. Schlichtkrull, Insulin crystals. II. Shape of rhombohedral zinc-insulin crystals in relation to species and crystallization media, 10 (9) (1956) 7.
[20] Controlled Release of Insulin Granules from PLGA Microparticles for Glucose Modulation in Diabetes. Primavera et al. Am. Diabetes Assoc. 67, supplement 1. page A595, 2279-PUB (2018).

What is claimed is:

1. A glucose-sensitive insulin-loaded microparticle, which comprises:
(a) a pH-sensitive polymeric porous matrix made of a biocompatible and biodegradable polymer,
(b) insulin granules encapsulated within the pH-sensitive polymeric porous matrix, and
(c) a blood glucose sensor attached to a surface of the glucose-sensitive insulin-loaded microparticle.

2. The glucose-sensitive insulin-loaded microparticle of claim 1, wherein the biocompatible and biodegradable polymer is selected from the group consisting of poly (lactic-co-glycolic) acid (PLGA), polyethylene glycol (PEG), hyaluronic acid, chitosan, polymethyl methacrylate, polylactic acid, polyglycolic acid, polycaprolactone, and any combination thereof.

3. The glucose-sensitive insulin-loaded microparticle of claim 1, wherein the blood glucose sensor is attached to an outer surface of the pH-sensitive polymeric porous matrix.

4. The glucose-sensitive insulin-loaded microparticle of claim 1, wherein the blood glucose sensor is selected from glucose oxidase and phenylboronic acid.

5. The glucose-sensitive insulin-loaded microparticle of claim 1, which is a microplate having a polygonal or substantially round base.

6. The glucose-sensitive insulin-loaded microparticle of claim 5, wherein the microplate has a polygonal base having an edge length of from 1 μm to 100 μm and wherein the microplate has a height of from 1 μm to 100 μm.

7. The glucose-sensitive insulin-loaded microparticle of claim 5, wherein the microplate has a polygonal base having an edge length of from 5 μm to 50 μm and wherein the microplate has a height of from 5 μm to 20 μm.

8. The glucose-sensitive insulin-loaded microparticle of claim 5, wherein the microplate has a polygonal base having an edge length of about 20 μm and wherein the microplate has a height of about 5 μm to 10 μm.

9. The glucose-sensitive insulin-loaded microparticle of claim 5, wherein the microplate has a substantially square or rectangular base.

10. The glucose-sensitive insulin-loaded microparticle of claim 1, wherein the insulin granules encapsulated within the pH-sensitive polymeric porous matrix have a diameter of from 50 nm to 500 nm.

11. The glucose-sensitive insulin-loaded microparticle of claim 10, wherein the insulin granules encapsulated within the pH-sensitive polymeric porous matrix have a diameter of from 100 nm to 200 nm.

12. A sustained drug delivery system comprising a plurality of glucose-sensitive insulin-loaded microparticles according to claim 1.

13. A method for controlling glucose levels in a subject suffering from diabetes, the method comprising releasing insulin from the sustained drug delivery system of claim 12 into blood of the subject.

14. The method of claim 13, wherein the diabetes is type-1 diabetes (T1D) or type-2 diabetes (T2D).

15. (canceled)

16. The method of claim 13, wherein the sustained drug delivery system releases the insulin in the blood of the subject over a prolonged period of time.
